(19) United States
(12) Patent Application Publication
(10) Pub. No.: US 2022/0244268 A1
(43) Pub. Date: Aug. 4, 2022

(54) METHOD FOR IDENTIFICATION AND ABSOLUTE QUANTIFICATION OF PRODUCT-RELATED IMPURITIES IN A PROTEIN USING HIGH-RESOLUTION MASS SPECTROMETRY

(71) Applicant: DR. REDDY'S LABORATORIES LIMITED, Hyderabad (IN)

(72) Inventors: Rakesh Komarla Sathyanarayana SETTY, East Bengaluru (IN); Murali JAYARAMAN, Kancheepuram (IN); Avinash BHARATI, Sirohi (IN); Vishal Balu SHRINEWAR, Mumbai (IN); Sakthi Deivanayagam CHANDINI, Chennai (IN)

(21) Appl. No.: 17/615,741

(22) PCT Filed: Jun. 3, 2020
(86) PCT No.: PCT/IN2020/050490

§ 371 Date: Dec. 1, 2021

(30) Foreign Application Priority Data
Jun. 6, 2019 (IN) 201941022458

(51) Int. Cl. G01N 33/68; G01N 30/86; G01N 30/72

(52) U.S. Cl. CPC G01N 33/6848; G01N 30/7233; G01N 30/8675

(57) ABSTRACT

The present invention discloses a method for the identification and absolute quantification of peptide based impurities in a protein/antibody composition using high resolution mass spectrometry. The method utilizes synthetic peptides for plotting a standard calibration curve which is, in turn, used for the absolute quantification of the impurities. In particular, the method is utilized for quantification of signal peptide remnants in heterogeneous unpurified or partially purified protein samples, comprising a complex mixture of proteins, with high sensitivity using unlabeled synthetic peptides.

### FIELD OF INVENTION

The invention relates to the detection, identification and quantification of impurities in proteins using mass spectrometry. In particular, the invention relates to methods using high-resolution mass spectrometry to detect, identify and quantitate the level of impurities in an antibody composition. More specifically, the invention relates to methods for detection, identification of signal peptide remnants in Fc-containing proteins such as antibodies and Fc-fusion proteins using high-resolution mass spectrometry coupled with liquid chromatography.

### BACKGROUND

Protein biopharmaceuticals have emerged as important therapeutics for the treatment of various diseases including cancer, cardiovascular diseases, diabetes, infection, autoimmune disorders etc. Especially, the introduction of recombinant antibodies and fusion proteins has changed the scenario of the healthcare industry.

Therapeutic antibodies and Fc-fusion proteins are usually produced in high-yield expression systems using stably transfected cell lines such as Chinese Hamster Ovary (CHO) cells. The resulting therapeutic proteins are complex glycoproteins with complicated post-translational modifications. The proteins thus produced possess heterogeneity, which can arise from the manufacturing process or from the product itself. Impurities arising from the process are known as ‘process-related impurities’ and include host cell DNA, host cell proteins, endotoxins, extractables and leachables used during purification, chromatographic resins, etc. ‘Product-related impurities’ are molecular variants of the biologic product such as sequence variants, acidic and basic variants, C-terminal and N-terminal variants, fragments, aggregates, etc. Impurities may influence the safety and efficacy of the therapeutic product.

Antibodies, including, Fc-fusion proteins, are initially synthesized in the cytoplasm of the cell in a precursor form with additional, N-terminal extension signal peptides. These signal peptides initiate the export of the protein and direct the transportation across membranes from the cytoplasm to non-cytoplasm sites in both eukaryotes and prokaryotes. The signal peptides are subsequently cleaved by signal peptidases during co-translational translocation, releasing the N-terminus of the mature secretory protein. Signal peptides, also called as leader sequence peptides, typically consist of 15 to 20 amino acid residues. The signal peptide is normally cleaved at a very specific site by signal peptidase after co-translocation of cytoplasmic proteins across the membrane. In some cases, however, there can be cleavage at non-specific sites, giving rise to N-terminal heterogeneity in the mature protein. These N-terminal variants are considered as sequence variants of the intended protein. These variants or signal peptide remnants are generally difficult to remove during downstream purification due to attributes being quite similar to the intended protein and, therefore, must be identified during or at an early stage of production to control and minimize their expression. The challenge of controlling signal peptide remnants in the final product is enhanced for biosimilars because of strict regulatory requirements.

Mass Spectrometry (MS) is one of the most widely used techniques for the detection, identification, characterization and quantification of impurities including sequence variants or signal peptide remnants. Impurity profiling and accurate quantification is critical for fully characterizing the biotherapeutic as the heterogeneity could lead to a dissimilar immunogenicity and safety profile of a biosimilar as compared with the reference drug and if detected timely, changes can be made in the upstream process at early stages to clear the respective impurities from the protein. The challenge is to detect and quantify trace levels of signal peptide remnants in a heterogeneous and complex protein mixture, for example in protein samples at the stage of clone selection and in early stages of the process, including cell culture harvest (i.e., unpurified) and partially purified samples thereof. Accordingly, there is a need for a method for detection and quantification of signal peptide remnants at an early stage of process development, allowing characterization of signal peptide remnants in a much more heterogeneous environment (such as stable clone pools, unpurified or partially purified mammalian cell cultured harvest samples) with high sensitivity.

The objective of the current invention is to address the above mentioned problem by providing a method for detection, identification and absolute quantification of a peptide base impurity viz., signal peptide remnants/sequence variants earlier in process development (clone selection stage) and in unpurified or partially purified samples containing therapeutic protein.

### SUMMARY

The present invention discloses a method for the detection, identification and quantification of the peptide based impurities viz., signal peptide remnants, in an Fc-containing protein composition, with high sensitivity using high-resolution mass spectrometry coupled to liquid chromatography. In particular, the method discloses the detection of signal peptide remnants in unpurified or partially purified samples of mammalian cell cultured therapeutic proteins using mass spectrometry or tandem mass spectrometry, and the use of unlabeled synthetic peptides for the identification of signal peptide remnants in the protein/antibody composition using retention time and isotopic spectral pattern of the native and the signal peptide remnants. After identification, the method provides for absolute quantification of the signal peptide remnants and the native peptides of unpurified or partially purified samples, with high sensitivity, employing a standard calibration curve plotted using different dilutions of the unlabeled synthetic peptides.

The method disclosed in the current invention can be used to detect and quantify signal peptide remnants in highly heterogeneous and complex protein mixtures, for example in samples from screening of different clones for selection of a stable clone and also during early stages of process development, such as in cell culture harvest or partially purified samples thereof.

### BRIEF DESCRIPTION OF DRAWINGS

FIG. 1 is an illustration of the calibration curve (cubic fit) generated in example 1 using known concentration of the synthetic standard of the native peptide.

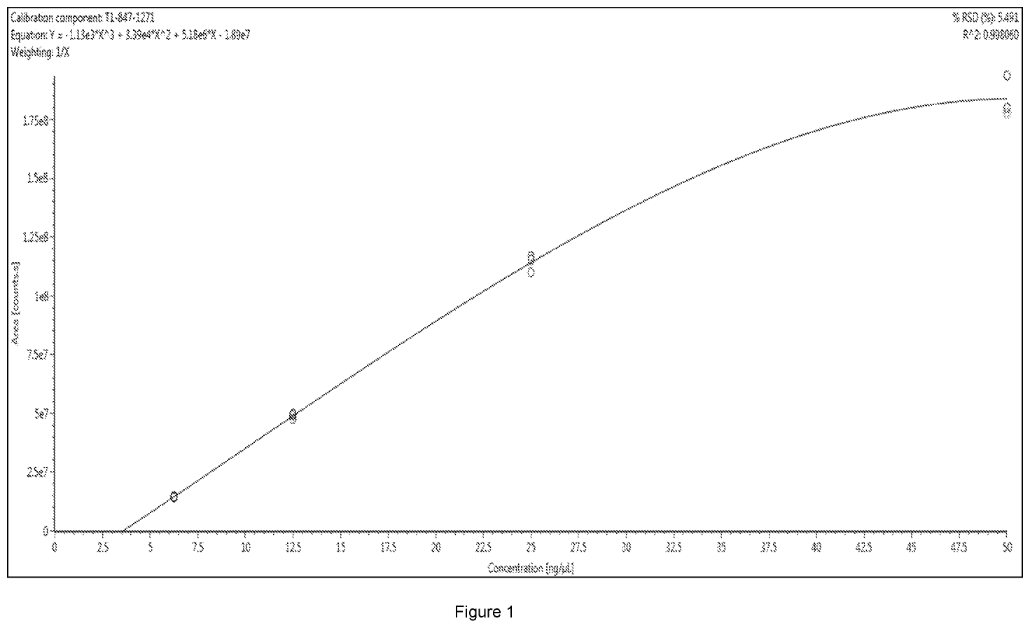

FIG. 2 is an illustration of the calibration curve (cubic fit) generated in example 1 using known concentration of the synthetic standard of the impurity peptide.

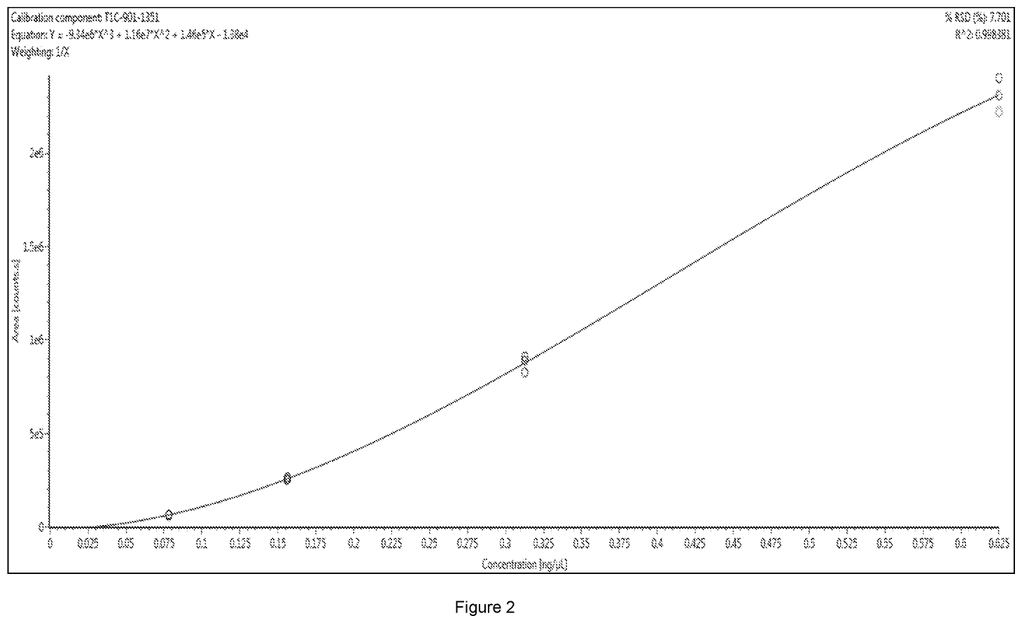

FIG. 3 is an illustration of the calibration curve plotted manually in example 2 for the synthetic standard of the native peptide on the basis of calculations shown in Table 15.

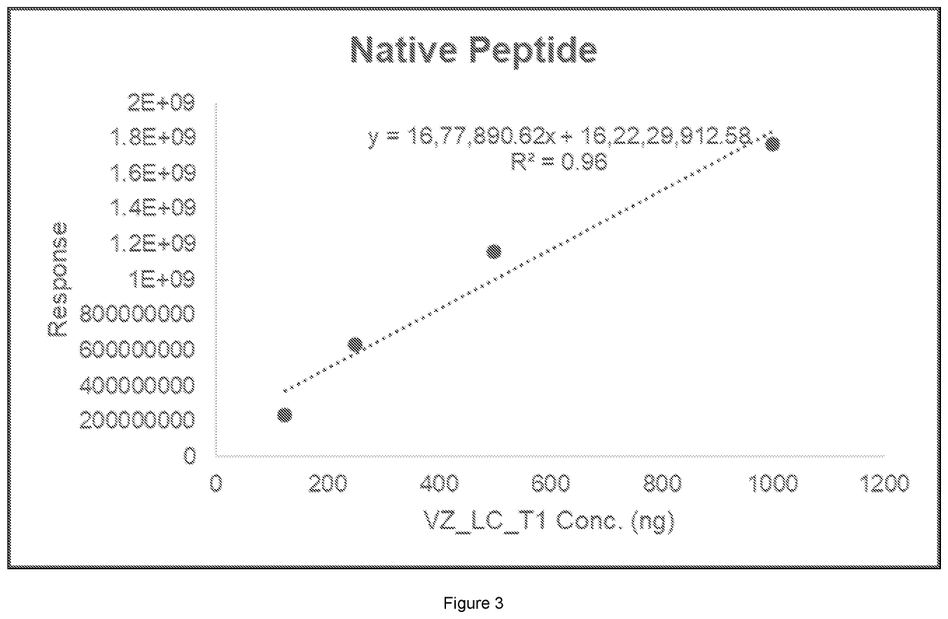

FIG. 4 is an illustration of the calibration curve plotted manually in example 2 for the synthetic standard of the impurity peptide on the basis of calculations shown in Table 16.

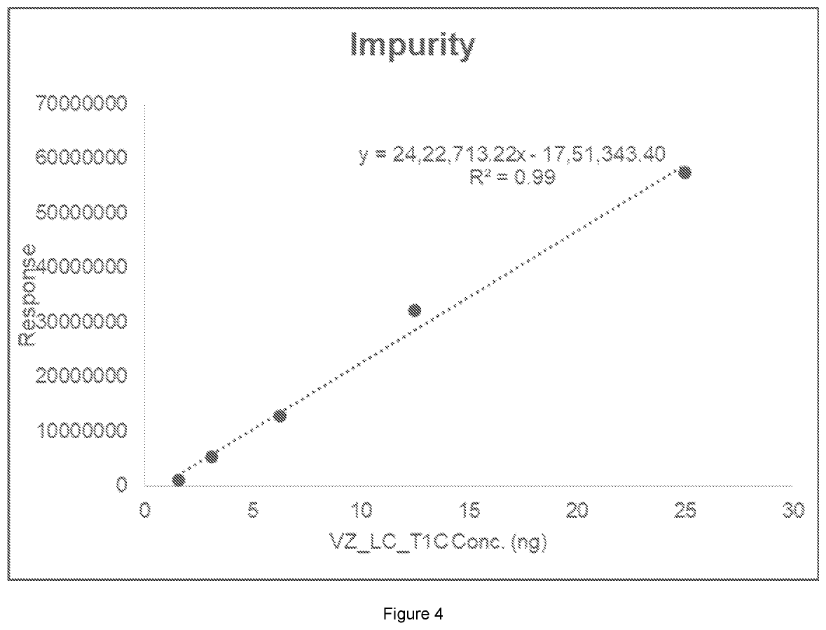

FIG. 5 is an illustration of the calibration curve plotted manually in example 3 for the synthetic standard of the native peptide on the basis of calculations shown in Table 18.

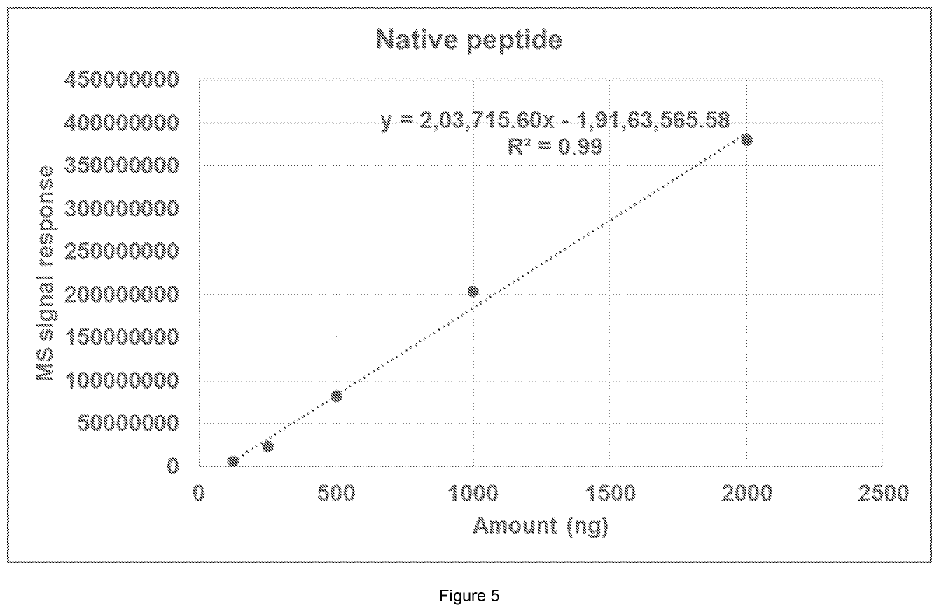

FIG. 6 is an illustration of the calibration curve plotted in example 3 for the synthetic standard of the impurity peptide (signal peptide remnant) on the basis of calculations shown in Table 19.

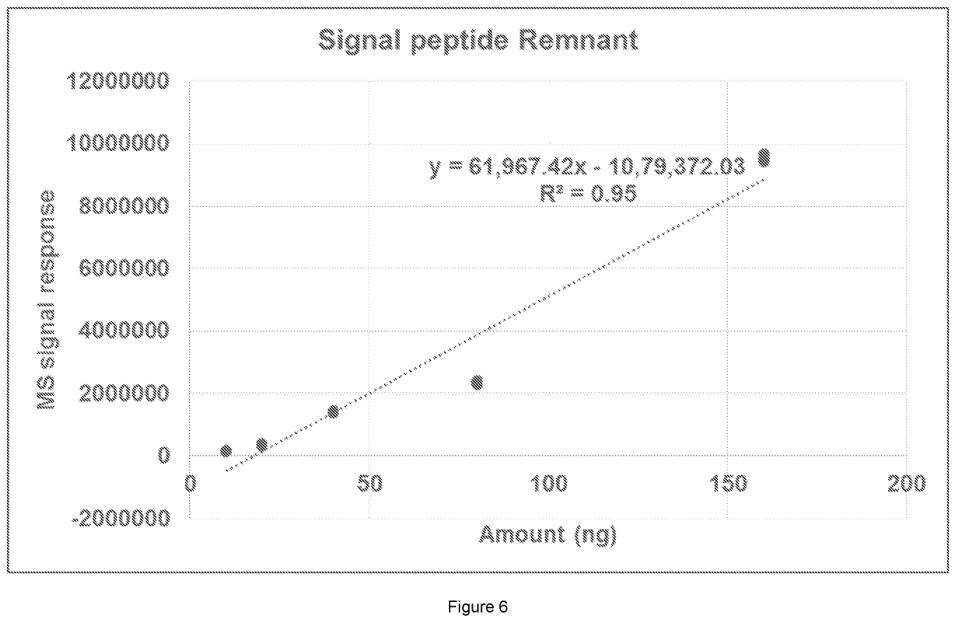

FIG. 7 is an illustration of the calibration curve plotted in example 4 for the synthetic standard of the native peptide on the basis of calculations shown in Table 21.

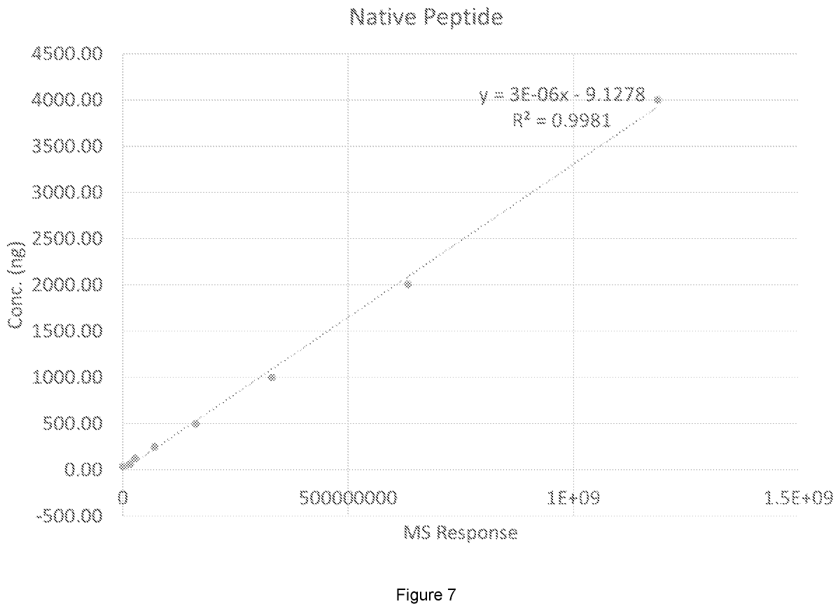

FIG. 8 is an illustration of the calibration curve plotted in example 4 for the synthetic standard of the impurity peptide (signal peptide remnant) on the basis of calculations shown in Table 22.

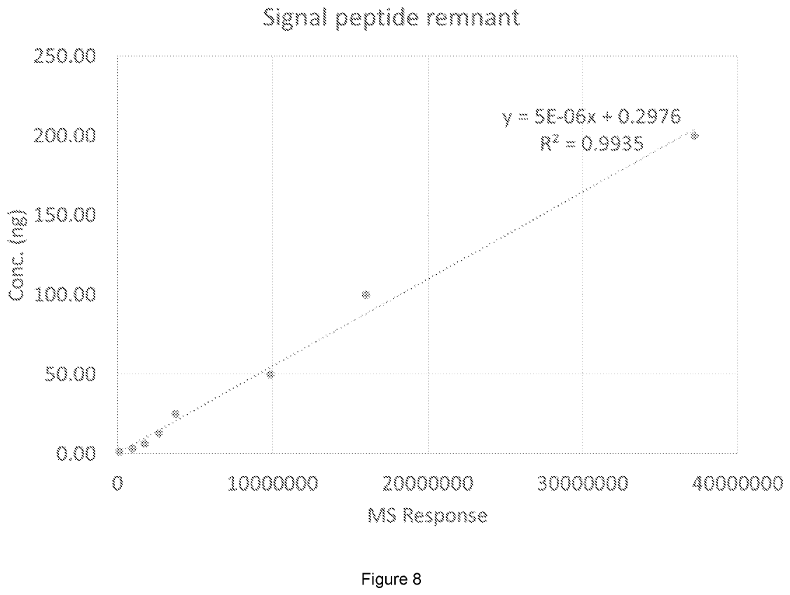

### DETAILED DESCRIPTION OF THE INVENTION

### Definitions

The term, “Fe-containing” proteins used herein denotes a protein that contains an Fc-region of an immunoglobulin. Examples of Fc containing proteins are antibodies, Fc-fusion proteins, etc.

The term “Fc-fusion protein” used herein is a protein that contains an Fc region fused or linked to a heterologous polypeptide. For instance, the heterologous polypeptide may be a ligand polypeptide, a receptor polypeptide, a hormone, cytokine, growth factor, an enzyme. Examples of Fc-fusion proteins are etanercept, abatacept, belatacept etc.

The term “antibody” as used herein encompasses whole antibodies and any antigen binding fragment (i.e., “antigen-binding portion”) or single chains or fusions thereof.

The term “glycoprotein” refers to protein or polypeptide having at least one glycan moiety. Thus, any polypeptide attached to a saccharide moiety is termed as glycoprotein.

The term “heterogeneous” as used herein refers to a protein sample which contains a mixture of proteins in addition to the target protein. Proteins other than the target protein include, but not limited to, host cell proteins, sequence variants, signal peptide remnants, charge variants, etc.

“Peptide based impurities” as used herein denotes peptides that have an amino acid sequence, which differ in identity from the peptide generated after proteolytic cleavage of the target protein by at least one amino acid. Peptide based impurities, for example, may include N-terminal signal peptide remnants, C-terminal extensions, sequence variants, charge variants, etc.

The term “signal peptide remnant” or “signal peptide variants” as used herein denotes an N-terminal peptide which results due to incomplete processing of the signal peptide and exists as an extension on the N-terminal of the otherwise completely processed mature protein.

“Unpurified protein composition”, as used herein denotes that the antibody/protein composition is obtained directly from the host cell organism or an expression system. For example, the composition may comprise harvested cell culture fluid.

The term “harvested cell culture fluid” or “cell culture harvest” as used herein denotes the fluid obtained directly from the host cell organism and comprises the target protein along with other contaminants such as host cell DNA, host cell proteins, etc. The cell culture fluid may be filtered or centrifuged to remove cells.

“Partially purified”, as used herein denotes that the antibody composition obtained from the host cell organism is subjected to one or more purification steps, such as filtration or affinity chromatography. Partially purified sample may still comprise a heterogeneous population of peptides such as host cell proteins, endotoxins, etc.

“Unlabeled” as used herein refers to the synthetic peptide homologous to the impurity or wild-type peptide which is free from the incorporation of any radioactive or non-radioactive isotope label.

“Native peptide” as used herein denotes a peptide having an amino acid sequence identity that is 100% similar to the amino acid sequence of the peptide generated by the proteolytic cleavage of a target protein.

“Reversed phase chromatography” is a chromatographic technique wherein mobile phase solute (e.g. proteins/peptides etc.) binds to an immobilized n-alkyl hydrocarbon or aromatic ligand via hydrophobic interaction. The biomolecules are then generally eluted using gradient elution instead of isocratic elution. While biomolecules are strongly adsorbed to the surface of a reversed phase matrix under aqueous/relatively less organic conditions, they desorb from the matrix within a very narrow window of organic/relatively increased organic modifier concentration. Since biomolecules would vary in terms of their hydrophobicity, it is an efficient technique to separate biomolecules by using gradient of organic modifier and thus pattern their separation

Mass spectrometry is an analytical technique that is used to identify unknown compounds, quantify known materials, and elucidate the structural and physical properties of ions. Mass Spectrometry can be used in conjunction with chromatography techniques, such as LC-MS and GC-MS. Examples of mass spectrometry tools for use as detection agents include, but are not limited to, electron ionisation (EI), chemical ionisation (CI), fast atom bombardment (FAB)/liquid secondary ionisation (LSIMS), matrix assisted laser desorption ionisation (MALDI), and electrospray ionisation (ESI). See, for example, Gary Siuzdak, Mass Spectrometry for Biotechnology, Academic Press, San Diego, 1996.

### DETAILED DESCRIPTION OF THE EMBODIMENTS

The present invention discloses a mass spectrometry based method for the detection, identification and ‘absolute’ quantification of peptide based impurities, in particular, signal peptide remnants in an Fc-containing protein composition using unlabeled synthetic peptides. Specifically, the method identifies and quantifies peptide remnants in partially purified or unpurified mammalian cell culture harvest samples. Whereas in the state-of-art, quantification of signal peptide remnants is done in purified samples with less complex peptide mixtures, and the quantification is ‘relative’, wherein the mass spectrometry response obtained for an impurity peptide is calculated relative to the total mass spectrometry response, as per the following formula:

$$Mass intensity response = \frac{MS response of impurity peptide}{Total MS response ( \begin{matrix} MS response of Native peptide + \\ MS response of impurity peptide\end{matrix} )} \times 100$$

In an embodiment, the invention discloses a method for identification and absolute quantification of a peptide based impurity in an Fc-containing protein composition, using mass spectrometry, wherein different dilutions of known concentrations of unlabeled synthetic peptides homologous to the impurity peptide and native peptide are used, followed by ionization and detection of the peptides using MS, followed by plotting an area versus concentration graph for the synthetic peptide and deducing the absolute amount of the native peptide and the impurity peptide using the graph thus plotted.

In the above embodiment, the Fc-containing protein composition is a mammalian cell culture harvest sample that is either unpurified, partially purified or purified.

In any of the above embodiment, the peptide based impurity is a signal peptide remnant of the Fc-containing protein.

In an embodiment, the invention discloses a method for identification and absolute quantification of a signal peptide remnant in an Fc-containing protein composition, comprising the steps of:
a) culturing the Fc-containing protein in a mammalian cell culture expression system
b) obtaining the Fc-containing protein mixture as a cell culture harvest
c) subjecting the protein mixture to proteolysis to generate fragments of the protein
d) subjecting the fragments to liquid chromatography followed by
e) ionization and detection of the fragments in MS
f) selective identification of the signal peptide remnants using unlabeled synthetic peptides by comparing the retention time and spectral distribution of the peptide mass of the synthetic peptides with the peptide in the protein/antibody sample
g) providing an unlabeled synthetic peptide, homologous to the signal peptide remnant and the native peptide, followed by preparing different dilutions of known concentration of the synthetic peptide,
h) subjecting different concentrations of the synthetic peptides to liquid chromatography followed by ionization and detection of the peptides in MS
i) plotting an area versus concentration graph for the synthetic peptide
j) deducing the absolute amount of the native peptide and the impurity peptide using the graph plotted in step (i)

In an embodiment, the invention discloses a method for identification and absolute quantification of signal peptide remnants in an Fc-containing protein composition, comprising the steps of:
a) denaturation of the protein sample,
b) reduction and alkylation of the protein sample,
c) proteolytic digestion of the protein with a protease for generating fragments of the protein (wherein the fragments include native peptides and signal peptide remnants),
d) subjecting the fragments to liquid chromatography followed by,
e) ionization and detection of the fragments in MS,
f) selective identification of the signal peptide remnants using unlabeled synthetic peptides (by comparing the retention time and spectral distribution of the peptide mass of the synthetic peptides with the peptide in the protein/antibody sample),
g) providing an unlabeled synthetic peptide, homologous to the signal peptide remnant, followed by preparing different dilutions of known concentration of the synthetic peptide,
h) subjecting different concentrations of the synthetic peptides to liquid chromatography followed by ionization and detection of the peptides in MS,
i) plotting an area versus concentration graph for the synthetic peptide,
j) deducing the absolute amount of the native peptide and the impurity peptide using the graph plotted in step (i), wherein the Fc-containing protein composition can be unpurified or partially purified.

In the above mentioned embodiment of the invention, the Fc-containing protein is a glycoprotein.

In the above mentioned embodiment of the invention, the protein is denatured using urea or guanidium hydrochloride and reduced using dithiothretriol (DTT).

In the above mentioned embodiment of the invention, the reduced protein is alkylated using iodoacetamide.

In the above mentioned embodiment of the invention, the protein is proteolytically digested using a protease, i.e., trypsin.

In the above mentioned embodiment, the digestion buffer used for reconstitution of the protease comprises 1 M Urea, 1 mM EDTA, 20 mM Hydroxyl ammonium chloride and 0.1 M Tris, and pH of the buffer is 7.5.

In an embodiment, the method disclosed in the invention can be used for the identification and absolute quantification of peptide based impurities in an antibody composition, including but not limited to sequence variants, N-terminal signal peptide remnants and charge variants.

In any of the above mentioned embodiments, the Fc-containing protein is a monoclonal antibody.

In the above mentioned embodiment, the antibody is a therapeutic antibody and is selected from the group consisting of anti-TNF-α antibody, anti-CTLA4 antibody, anti-PD1 antibody, anti-PDL1 antibody, anti-Her2 antibody, anti-IL6R antibody, anti-VEGFR antibody, anti-IL17A antibody, Anti-α4β7 antibody, and anti-IgE antibody.

In any of the above mentioned embodiments, the Fc-containing protein is an Fc-fusion protein.

In any of the above mentioned embodiments, the Fc-fusion protein is selected from the group consisting of etanercept, abatacept, belatacept, alefacept, and aflibercept.

In the above mentioned embodiments, liquid chromatography is the technique used to separate the peptides generated after treatment with the protease. Further, the chromatography is reversed-phase chromatography.

In an embodiment of the invention, the identification and quantification step for impurities can be preceded by a detection step wherein the impurities are detected using mass spectrometry or tandem mass spectrometry.

In an embodiment of the invention, the method disclosed is capable of detecting signal peptide remnants up to less than 1 ng/μl of the sample.

In an embodiment of the invention, the method disclosed is capable of detecting the signal peptide remnants up to a level of 0.08 ng/μl of the sample.

In an embodiment, the disclosed method is employed in the early stages of product development (for example, screening of different clones) and for monitoring the level of signal peptide remnants at different stages of the purification process (for example, in affinity chromatography eluate, in ion-exchange chromatography eluate, in drug substance, etc.).

In an embodiment, the method is capable of identifying specific signal peptide remnants from a complex mixture of peptides.

In an embodiment, the method disclosed in the invention confidently and accurately identifies and quantifies even trace-level of signal peptide remnants in a heterogeneous protein sample containing a complex mixture of peptides.

Specific embodiments of the invention are more fully defined by reference to the following examples. These examples should not, however, be construed as limiting the scope of the invention.

### EXAMPLES

### Example 1

Sample monoclonal antibody (mAb 1) was used for the development of the method. mAb1 expressed in a host cell line and harvested from the cell culture extract is partially purified (viz., subjected to filtration and/or chromatography) and concentrated. 1 mg of mAb1 was mixed with denaturation buffer (8.2 M Guanidium HCl, 1 mM EDTA and 0.1 M Tris, pH 7.5) to get final concentration of the protein to 1 mg/ml. After mixing, the sample was kept at room temperature for few minutes. Post that, the denatured sample was reduced by addition of 5 mM DTT and incubated at 37° C. for 10 minutes to reduce inter-chain and intra-chain disulfide bonds to produce HC (heavy chain) and LC (light chain) molecules. The reduced protein sample was alkylated by addition of 10 mM concentration of iodoacetamide and incubated at room temperature for 40 minutes. Further, the sample cleanup was performed using PD-10 cartridges to remove salts, excipients, buffer components and denaturing agents. The cleaned up sample was treated with trypsin (enzyme:protein ratio 1:50 w/w) and incubated at 37° C. for 17 h. The composition of digestion buffer used for reconstitution of trypsin was −1 M Urea, 1 mM EDTA, 20 mM Hydroxyl ammonium chloride and 0.1 M Tris, and pH 7.5.

Post incubation with Trypsin, the reaction mixture of the protease was subjected to RP-UPLC using 2.1 mm×150 mm ACQUITY UPLC™ BEH C8 Column 1.7 μm particle size, 300 Å pore size (Waters ACQUITY UPLC™ H Class Bio). The operating parameters and the mobile phase gradient used during reverse phase chromatography are provided in Table 1 and Table 2, respectively. The eluate from RP-UPLC was then subjected to MS using Waters Xevo G2-XS HDMS instrument. Data was analyzed using the UNIFI™ software. The impurities (signal peptide remnants) were first detected using the UNIFI™ software based on the masses of respective peptides. The critical parameters for mass spectrometer are given in Table 3.

TABLE 1
Operating parameters for reversed-phase UPLC

| Sr. No. | Parameter name | Value/ranges |
|---|---|---|
| 1 | Column Temperature | 60° C. |
| 2 | Injection volume | 20 μL |
| 3 | Detection wave length | 214 nm and 280 nm |
| 4 | Mobile phase A | Water |
| 5 | Mobile phase B | Acetonitrile |
| 6 | Mobile phase C | 1.0% Formic Acid in water |

TABLE 2
Mobile phase gradient used for reverse phase chromatography

| Time | | | | Flow rate |
|---|---|---|---|---|
| (min) | % A | % B | % C | (mL/min) |
| 0 | 87 | 3 | 10 | 0.3 |
| 0.33 | 87 | 3 | 10 | 0.3 |
| 5.33 | 78 | 12 | 10 | 0.2 |
| 10.67 | 70 | 20 | 10 | 0.3 |
| 20.33 | 50 | 40 | 10 | 0.3 |
| 21.33 | 10 | 80 | 10 | 0.3 |
| 22.67 | 10 | 80 | 10 | 0.3 |
| 22.73 | 87 | 3 | 10 | 0.3 |
| 25.00 | 87 | 3 | 10 | 0.3 |

TABLE 3
MS method operating parameters

| Sr. No. | MS method parameters | Value | |
|---|---|---|---|
| 1 | Mass range | 50-1995 m/z | |
| 2 | Mode | Sensitivity | |
| 3 | Polarity | Positive | |
| 4 | Acquisition time | 0 min to 25 min | |
| 5 | Scan time | 1 | sec |
| 6 | Capillary voltage | 3 | kV |
| 7 | Sampling cone voltage | 25 | V |
| 8 | Collision energy (Low) | 6 | eV |
| 9 | Collision energy (High) | 30 to 60 eV | |
| 10 | Source Temperature | 120° C. | |
| 11 | Cone gas | 50 | L/H |
| 12 | Desolvation gas | 600 | L/H |

| | | |
|---|---|---|
| 13 | Desolvation temperature | 300° C. |

The procedure for preparation of dilutions of the native and impurity peptide standards is shown in Table 4 and Table 5, respectively.

TABLE 4
Preparation of native peptide standard dilutions
Concentration of Native synthetic peptide stock (Master stock): 1000 ng/uL

| | | | Conc. of | Conc. Of | | Volume | | |
|---|---|---|---|---|---|---|---|---|
| | | | working | working | Master | of | Volume | Volume |
| | Conc. | Injection | stock | stock | Stock | Master | of | of 500 |
| | (ng) on | volume | (1X) | (2X) | used | stock | buffer | mM IAM |
| Sr. No. | column | (μL) | (ng/μL) | (ng/μL) | (ng/μL) | (μL) | (μL) | (μL) |
| 1 | 2000 | 20 | 100 | 200 | 1000 | 40 | 156 | 4 |
| 2 | 1000 | 20 | 50 | 100 | 200 | 100 | 100 | 0 |
| 3 | 500 | 20 | 25 | 50 | 100 | 100 | 100 | 0 |
| 4 | 250 | 20 | 12.5 | 25 | 50 | 100 | 100 | 0 |
| 5 | 125 | 20 | 6.25 | 12.5 | 25 | 100 | 100 | 0 |

TABLE 5
Preparation of impurity peptide standard dilutions
Concentration of impurity synthetic peptide stock (Master stock): 1000 ng/uL

| | | | Conc. Of | Conc. Of | | Volume | | Volume |
|---|---|---|---|---|---|---|---|---|
| | | | working | working | Master | of | Volume | of 500 |
| | Conc. | Injection | stock | stock | Stock | Master | of | mM |
| | (ng) on | volume | (1X) | (2X) | used | stock | buffer | IAM |
| Sr. No. | column | (μL) | (ng/μL) | (ng/μL) | (ng/μL) | (μL) | (μL) | (μL) |
| 1 | 25 | 20 | 1.25 | 2.5 | 1000 | 2.5 | 977.5 | 20 |
| 2 | 12.5 | 20 | 0.625 | 1.25 | 2.5 | 100 | 100 | 0 |
| 3 | 6.25 | 20 | 0.3125 | 0.625 | 1.25 | 100 | 100 | 0 |
| 4 | 3.125 | 20 | 0.15625 | 0.3125 | 0.625 | 100 | 100 | 0 |
| 5 | 1.5625 | 20 | 0.078125 | 0.15625 | 0.3125 | 100 | 100 | 0 |

The quantitative processing parameters used in the software have been shown in Tables 6, 7, and 8. Note that Extraction Ion Chromatogram is abbreviated as XIC and “(2)” means signal response from two mass values in Table 6.

TABLE 6
Component list

| Component | Expected | Extraction | Expected | Calibration | Extraction |
|---|---|---|---|---|---|
| name | RT (min) | window (min) | m/z | response factor | mode |
| T1-847-1271 | 13.71 | 1 | 847.7611, | 1 | XIC (2) |
| | | | 1271.13801 | | |
| T1C-901-1351 | 13.55 | 1 | 1351.15333, | 1 | XIC (2) |
| | | | 901.10465 | | |

TABLE 7
Default amount of components at different concentrations

| Component name | Level 1 | Level 2 | Level 3 | Level 4 |
|---|---|---|---|---|
| T1-847-1271 | 6.25 | 12.5 | 25 | 50 |
| T1C-901-1351 | 0.078125 | 0.15625 | 0.3125 | 0.625 |

TABLE 8
Processing and calibration parameters used in the software
Processing parameters

| | |
|---|---|
| Mass tolerance | 100 ppm |

Calibration parameters

| | |
|---|---|
| Calibration curve type | Cubic |
| Weight type | 1/X |
| Component value type | Concentration |
| Component value units | ng/μL |
| Compute calibration points by averaging | None |

Table 9 shows the calculation of relative percentage of impurity using absolute quantification.

TABLE 9
Calculation of relative percentage of impurity using absolute quantification

| | | | | | Total | |
|---|---|---|---|---|---|---|
| | | | | | amount | Final |
| | | Type of | Calculated | Average | (Native and | Percentage |
| Sample | Preparation | Peptide | amount (ng) | amount (ng) | Impurity) (ng) | (%) |
| mAb1 | 1 | Native | 411.1 | 404.1 | 405.5 | 99.65 |
| (partially | 2 | Native | 397.0 | | | |
| purified) | | | | | | |
| mAb1 | 1 | Impurity | 1.4 | 1.4 | | 0.35 |
| (partially | 2 | Impurity | 1.5 | | | |
| purified) | 2 | Impurity | 0.6 | | | |

### Example 2

Sample monoclonal antibody (mAb1), as described in example 1 was used for the development of the method. 1 mg of mAb1 was mixed with denaturation buffer (8.2 M Guanidium HCl, 1 mM EDTA and 0.1 M Tris, pH 7.5) to get final concentration of the protein to 1 mg/ml. After mixing, the sample was kept at room temperature for few minutes. Post that, the denatured sample was reduced by addition of 5 mM DTT and incubated at 37° C. for 10 minutes to reduce inter-chain and intra-chain disulfide bonds to produce HC and LC molecules. The reduced protein sample was alkylated by addition of 10 mM concentration of iodoacetamide and incubated at room temperature for 40 minutes. Further, the sample cleanup was performed using PD-10 cartridges to remove salts, excipients, buffer components and denaturing agents. The cleaned up sample was treated with trypsin (enzyme:protein ratio 1:50) and incubated at 37° C. for 17 h. The composition of digestion buffer used for reconstitution of trypsin was −1 M Urea, 1 mM EDTA, 20 mM Hydroxyl ammonium chloride and 0.1 M Tris, and pH 7.5.

Post incubation with Trypsin, the reaction mixture of the protease was subjected to RP-UPLC using 2.1 mm×150 mm ACQUITY BEH C8 Column 1.7 μm particle size, 300 Å pore size (Waters ACQUITY UPLC H Class Bio). The operating parameters and the mobile phase gradient used during reverse phase chromatography are provided in Table 10 and Table 11, respectively. The eluate from RP-UPLC was then subjected to MS using Waters Xevo G2-XS HDMS instrument. The impurities (signal peptide remnants) were first detected using the UNIFI™ software based on the masses of respective peptides. The critical parameters for mass spectrometer are given in Table 12.

TABLE 10
Operating parameters for reversed-phase UPLC

| Sr. No. | Parameter name | Value/ranges |
|---|---|---|
| 1 | Column Temperature | 60° C. |
| 2 | Injection volume | 20 μL |
| 3 | Detection wave length | 214 nm and 280 nm |
| 4 | Mobile phase A | Water |
| 5 | Mobile phase B | Acetonitrile |
| 6 | Mobile phase C | 1.0% Formic Acid in water |

TABLE 11
Mobile phase gradient used for reverse phase chromatography

| | Time | | | | Flow rate |
|---|---|---|---|---|---|
| Sr. No. | (min) | % A | % B | % C | (mL/min) |
| 1 | 0 | 87 | 3 | 10 | 0.3 |
| 2 | 0.33 | 87 | 3 | 10 | 0.3 |
| 3 | 5.33 | 78 | 12 | 10 | 0.2 |
| 4 | 10.67 | 70 | 20 | 10 | 0.3 |
| 5 | 20.33 | 50 | 40 | 10 | 0.3 |
| 6 | 21.33 | 10 | 80 | 10 | 0.3 |
| 7 | 22.67 | 10 | 80 | 10 | 0.3 |
| 8 | 22.73 | 87 | 3 | 10 | 0.3 |
| 9 | 25.00 | 87 | 3 | 10 | 0.3 |

TABLE 12
MS method operating parameters

| MS method Parameters | Value | |
|---|---|---|
| Mass range | 50-1995 m/z | |
| Mode | Sensitivity | |
| Polarity | Positive | |
| Acquisition time | 0 min to 25 min | |
| Scan time | 1 | sec |
| Capillary voltage | 3 | kV |
| Sampling cone voltage | 25 | V |
| Collision energy (Low) | 6 | eV |
| Collision energy (High) | 30 to 60 eV | |
| Source Temperature | 120° C. | |
| Cone gas | 50 | L/H |
| Desolvation gas | 600 | L/H |

| | |
|---|---|
| Desolvation temperature | 300° C. |

The procedure for preparation of dilutions of the native and impurity peptide standards is shown in Table 13 and Table 14, respectively.

TABLE 13
Preparation of native peptide standard dilutions
Conc. of LC_T1 synthetic peptide stock (Master stock): 1000 ng/uL

| | | | Conc. Of | Conc. Of | | Volume | | Volume |
|---|---|---|---|---|---|---|---|---|
| | | | working | working | Master | of | Volume | of 500 |
| | Conc. | Injection | stock | stock | Stock | Master | of | mM |
| | (ng) on | volume | (1X) | (2X) | used | stock | buffer | IAM |
| Sr. No. | column | (μL) | (ng/μL) | (ng/μL) | (ng/μL) | (μL) | (μL) | (μL) |
| 1 | 2000 | 20 | 100 | 200 | 1000 | 40 | 156 | 4 |
| 2 | 1000 | 20 | 50 | 100 | 200 | 100 | 100 | 0 |
| 3 | 500 | 20 | 25 | 50 | 100 | 100 | 100 | 0 |
| 4 | 250 | 20 | 12.5 | 25 | 50 | 100 | 100 | 0 |
| 5 | 125 | 20 | 6.25 | 12.5 | 25 | 100 | 100 | 0 |

TABLE 14
Preparation of impurity peptide standard dilutions
Conc. of LC_T1C synthetic peptide stock (Master stock): 1000 ng/uL

| | | | Conc. Of | Conc. Of | | Volume | | Volume |
|---|---|---|---|---|---|---|---|---|
| | | | working | working | Master | of | Volume | of 500 |
| | Conc. | Injection | stock | stock | Stock | Master | of | mM |
| | (ng) on | volume | (1X) | (2X) | used | stock | buffer | IAM |
| Sr. No. | column | (μL) | (ng/μL) | (ng/μL) | (ng/μL) | (μL) | (μL) | (μL) |
| 1 | 25 | 20 | 1.25 | 2.5 | 1000 | 2.5 | 977.5 | 20 |
| 2 | 12.5 | 20 | 0.625 | 1.25 | 2.5 | 100 | 100 | 0 |
| 3 | 6.25 | 20 | 0.3125 | 0.625 | 1.25 | 100 | 100 | 0 |
| 4 | 3.125 | 20 | 0.15625 | 0.3125 | 0.625 | 100 | 100 | 0 |
| 5 | 1.5625 | 20 | 0.078125 | 0.15625 | 0.3125 | 100 | 100 | 0 |

The standards were injected on LC-MS in triplicates. A manual method was utilized for data analysis and quantification. The details of the MS response for the native peptide are captured in Table 15.

TABLE 15
Manual calculations used for plotting calibration curve of the synthetic native peptide standard

| | Std. Conc. | Response | | | | | % |
|---|---|---|---|---|---|---|---|
| Sr. No. | (ng) | Inj 1 | Inj 2 | Inj 3 | Average | SD | RSD |
| 1 | 125 | 240843440 | 237955776 | 232278880 | 237026032 | 4357320.269 | 1.8 |
| 2 | 250 | 639149632 | 637769408 | 626731456 | 634550165 | 6806277.713 | 1.1 |
| 3 | 500 | 1157663360 | 1166446464 | 1150037760 | 1.158E+09 | 8211153.575 | 0.7 |
| 4 | 1000 | 1769960448 | 1760135680 | 1765921408 | 1.765E+09 | 4938193.983 | 0.3 |

Table 16 shows the calculations done manually for plotting the calibration curve for synthetic peptide standard of impurity

TABLE 16
Manual calculations used for plotting calibration
curve of the synthetic peptide standard of impurity

| | Std Conc. | Response | | | | | % |
|---|---|---|---|---|---|---|---|
| Sr. No. | (ng) | Inj 1 | Inj 2 | Inj 3 | Average | SD | RSD |
| 1 | 1.5625 | 1159891 | 1141724 | 1141646 | 1147753.7 | 10511.31135 | 0.9 |
| 2 | 3.125 | 3659736 | 6161821 | 5948955 | 5256837.3 | 1387219.336 | 26.4 |
| 3 | 6.25 | 13257732 | 11413901 | 13710262 | 12793965 | 1216399.742 | 9.5 |
| 4 | 12.5 | 31667276 | 32317372 | 32238636 | 32074428 | 354794.8742 | 1.1 |
| 5 | 25 | 57969412 | 57044288 | 56947712 | 57320471 | 564070.3604 | 1.0 |

The absolute amount of both native peptide and the impurity was calculated using the calibration curves of the synthetic peptide standards—both native and impurity peptide (Table 17).

TABLE 17
Calculation of relative percentage of impurity using absolute
quantification

| | | Calculated | Total amount of | |
|---|---|---|---|---|
| | Type of | amount | (Native and | Final |
| Sample | Peptide | (ng) | Impurity) in ng | percentage (%) |
| mAb1 | Native | 626.123 | 628.557 | 99.61 |
| | Impurity | 2.434 | | 0.39 |

### Example 3

The method as disclosed herein was used to identify and quantify signal peptide remnants in a sample monoclonal antibody (mAb 2). mAb2 expressed in a host cell line and harvested from the cell culture extract is partially purified (viz., subjected to filtration and/or chromatography) and concentrated. 1 mg of partially purified sample of mAb2 was mixed with denaturation buffer (8.2 M Guanidium HCl, 1 mM EDTA and 0.1 M Tris, pH 7.5) to get final concentration of the protein to 1 mg/ml. After mixing, the sample was kept at room temperature for few minutes. Post that, the denatured sample was reduced by addition of 5 mM DTT and incubated at 37° C. for 30 minutes to reduce inter-chain and intra-chain disulfide bonds to produce HC (heavy chain) and LC (light chain) molecules. The reduced protein sample was alkylated by addition of 6.5 mM concentration of iodoacetamide and incubated at room temperature for 40 minutes. Further, the sample cleanup was performed using PD-10 cartridges to remove salts, excipients, buffer components and denaturing agents. The cleaned up sample was treated with trypsin (enzyme:protein ratio 1:50 w/w) and incubated at 37° C. for 17 hrs. The composition of digestion buffer used for reconstitution of trypsin was −1 M Urea, 1 mM EDTA, 20 mM Hydroxyl ammonium chloride and 0.1 M Tris, and pH 7.5.

Post incubation with trypsin, the reaction mixture of the protease was subjected to RP-UPLC using 2.1 mm×50 mm ACQUITY UPLC™ BEH C8 Column 1.7 μm particle size, 300 Å pore size (Waters ACQUITY UPLC™ H Class Bio). The operating parameters and the mobile phase gradient used during reversed phase chromatography are provided in Tables 10 and 11, respectively. The eluate from RP-UPLC was then subjected to MS using Waters Xevo G2-XS HDMS instrument. Data was analyzed using the UNIFI™ software. The signal peptide remnants were first detected using the UNIFI™ software based on the masses of respective peptides. The critical parameters used for mass spectrometer are given in Table 12.

Dilutions of known concentration of synthetic peptide standards were prepared and injected on LC-MS in triplicates. Tables 18 and 19, respectively, show the MS response obtained for various dilutions of the native peptide and signal peptide remnant standard.

TABLE 18
Calculations used for plotting calibration curve of the synthetic native peptide standard

| | Std Conc. | Response | | | | | % |
|---|---|---|---|---|---|---|---|
| Sr. No. | (ng) | Inj 1 | Inj 2 | Inj 3 | Average | SD | RSD |
| 1 | 125 | 5248050 | 5405304 | 5611819 | 5421724.333 | 182439.557 | 3.36 |
| 2 | 250 | 22708480 | 22648192 | 23011908 | 22789526.667 | 194932.690 | 0.86 |
| 3 | 500 | 81756144 | 81036808 | 82287904 | 81693618.667 | 627887.222 | 0.77 |
| 4 | 1000 | 203093728 | 203307344 | 203826080 | 203409050.667 | 376620.562 | 0.19 |
| 5 | 2000 | 380354112 | 379748544 | 380695968 | 380266208.000 | 479789.948 | 0.13 |

TABLE 19
Calculations used for plotting calibration curve of the
synthetic peptide standard of signal peptide remnant

| | Std Conc. | Response | | | | | % |
|---|---|---|---|---|---|---|---|
| Sr. No. | (ng) | Inj 1 | Inj 2 | Inj 3 | Average | SD | RSD |
| 1 | 5 | 22276 | 21848 | 21565 | 21896.333 | 357.956 | 1.63 |
| 2 | 10 | 164363 | 140056 | 144831 | 149750.000 | 12878.470 | 8.60 |
| 3 | 20 | 380165 | 317780 | 391682 | 363209.000 | 39761.864 | 10.95 |
| 4 | 40 | 1419418 | 1454486 | 1390554 | 1421486.000 | 32016.131 | 2.25 |
| 5 | 80 | 2299494 | 2340565 | 2399166 | 2346408.333 | 50092.268 | 2.13 |
| 6 | 160 | 9417899 | 9673331 | 9505332 | 9532187.333 | 129816.345 | 1.36 |

The absolute amount of both native peptide and the signal peptide remnant was calculated using the calibration curves of the synthetic peptide standards—both native and signal peptide remnant (Table 20).

TABLE 20
Calculation of relative percentage of signal peptide remnant
using absolute quantification

| | Type of | Calculated | Total amount | Percentage |
|---|---|---|---|---|
| Sample | Peptide | amount (ng) | (ng) | (%) |
| Clone 1 | Native | 877.1202087 | 995.22745 | 88.1 |
| | Remnant | 118.1072414 | | 11.9 |
| Clone 2 | Native | 884.396863 | 1018.284025 | 86.9 |
| | Remnant | 133.8871625 | | 13.1 |
| Clone 3 | Native | 895.8897482 | 990.6581447 | 90.4 |
| | Remnant | 94.76839652 | | 9.6 |
| Clone 4 | Native | 907.9130984 | 1030.964873 | 88.1 |
| | Remnant | 123.0517751 | | 11.9 |
| Clone 5 | Native | 912.3974677 | 1009.082936 | 90.4 |
| | Remnant | 96.68546843 | | 9.6 |
| Clone 6 | Native | 970.9713619 | 1148.307388 | 84.6 |
| | Remnant | 177.3360264 | | 15.4 |

### Example 4

The method disclosed herein is used to quantify signal peptide remnants in drug substance (DS) and in-process samples of an Fc-fusion protein (FP-1). FP-1 expressed in a host cell line and harvested from the cell culture extract is partially purified (viz., subjected to filtration and/or chromatography) and concentrated. 1 mg of FP-1 was mixed with denaturation buffer (8.2 M Guanidium HCl, 1 mM EDTA and 0.1 M Tris, pH 7.5) to get final concentration of the protein to 1 mg/ml. After mixing, the sample was kept at room temperature for few minutes. Post that, the denatured sample was reduced by addition of 5 mM DTT and incubated at 37° C. for 30 minutes to reduce inter-chain and intra-chain disulfide bonds to produce HC (heavy chain) and LC (light chain) molecules. The reduced protein sample was alkylated by addition of 6.5 mM concentration of iodoacetamide and incubated at room temperature for 40 minutes. Further, the sample cleanup was performed using PD-10 cartridges to remove salts, excipients, buffer components and denaturing agents. The cleaned up sample was treated with trypsin (enzyme:protein ratio 1:50 w/w) and incubated at 37° C. for 17 hrs. The composition of digestion buffer used for reconstitution of trypsin was −1 M Urea, 1 mM EDTA, 20 mM Hydroxyl ammonium chloride and 0.1 M Tris, and pH 7.5.

Post incubation with Trypsin, the reaction mixture of the protease was subjected to RP-UPLC using 2.1 mm×50 mm ACQUITY UPLC™ BEH C8 Column 1.7 μm particle size, 300 Å pore size (Waters ACQUITY UPLC™ H Class Bio). The operating parameters and the mobile phase gradient used during reverse phase chromatography are provided in Tables 10 and 11, respectively. The eluate from RP-UPLC was then subjected to MS using Waters Xevo G2-XS HDMS instrument. Data was analyzed using the UNIFI™ software. The impurities (signal peptide remnants) were first detected using the UNIFI™ software based on the masses of respective peptides. The critical parameters for mass spectrometer are given in Table 12.

Dilutions of known concentration of synthetic peptide standards were prepared and injected on LC-MS in triplicates. Tables 21 and 22, respectively, show the MS response obtained for various dilutions of the native peptide and signal peptide remnant standard.

TABLE 21
Calculations used for plotting calibration curve of the synthetic native peptide standard

| | Std Conc. | Response | | | | | % |
|---|---|---|---|---|---|---|---|
| Sr. No. | (ng) | Inj 1 | Inj 2 | Inj 3 | Average | SD | RSD |
| 1 | 31.25 | 115400 | 153867 | 150797 | 140021.333 | 21377.880 | 15.27 |
| 2 | 62.5 | 14993804 | 15340880 | 15745853 | 15360179.000 | 376395.753 | 2.45 |
| 3 | 125 | 26735270 | 27100136 | 26915104 | 26916836.667 | 182439.171 | 0.68 |
| 4 | 250 | 70762024 | 70043496 | 69542832 | 70116117.333 | 612831.687 | 0.87 |
| 5 | 500 | 164345120 | 159095344 | 164292640 | 162577701.333 | 3015924.068 | 1.86 |
| 6 | 1000 | 328673280 | 331684640 | 331287616 | 330548512.000 | 1636086.277 | 0.49 |
| 7 | 2000 | 638299136 | 630660288 | 628509312 | 632489578.667 | 5144889.867 | 0.81 |
| 8 | 4000 | 1194677504 | 1184736384 | 1183331584 | 1187581824.000 | 6185052.284 | 0.52 |

TABLE 22
Calculations used for plotting calibration curve of the
synthetic peptide standard of signal peptide remnant

| | Std Conc. | Response | | | | | % |
|---|---|---|---|---|---|---|---|
| Sr. No. | (ng) | Inj 1 | Inj 2 | Inj 3 | Average | SD | RSD |
| 1 | 1.56 | 123000 | 122000 | 121000 | 122000.000 | 1000.000 | 0.82 |
| 2 | 3.13 | 965000 | 945000 | 956000 | 955333.333 | 10016.653 | 1.05 |
| 3 | 6.25 | 1790000 | 1740000 | 1740000 | 1756666.667 | 28867.513 | 1.64 |
| 4 | 12.5 | 2690000 | 2690000 | 2660000 | 2680000.000 | 17320.508 | 0.65 |
| 5 | 25 | 3770000 | 3700000 | 3720000 | 3730000.000 | 36055.513 | 0.97 |
| 6 | 50 | 9810000 | 9830000 | 9870000 | 9836666.667 | 30550.505 | 0.31 |
| 7 | 100 | 16300000 | 15900000 | 15900000 | 16033333.333 | 230940.108 | 1.44 |
| 8 | 200 | 37800000 | 37300000 | 36500000 | 37200000.000 | 655743.852 | 1.76 |

The absolute amount of both native peptide and the signal peptide remnant was calculated using the calibration curves of the synthetic peptide standards—both native and signal peptide remnant (Table 23).

TABLE 23
Calculation of relative percentage of signal peptide remnant using
absolute quantification

| | Type of | Calculated | Total amount | Percentage |
|---|---|---|---|---|
| Sample | Peptide | amount (ng) | (ng) | (%) |
| Sample 1 | Native | 1294.94 | 1300.10 | 99.60 |
| (partially | Remnant | 5.16 | | 0.40 |
| purified) | | | | |
| Sample 2 | Native | 1406.98 | 1410.97 | 99.72 |
| (DS) | Remnant | 3.99 | | 0.28 |

From the above examples, it can be concluded that the method disclosed in the invention can be used to identify and quantify signal peptide remnants in heterogeneous and complex, partially purified samples of antibodies and Fc-fusion proteins. This is particularly useful for manufacturing of antibody and Fc-fusion protein compositions at an industrial scale.

1. A method for identification and absolute quantification of a peptide based impurity in an Fc-containing protein composition using mass spectrometry, the method comprising the following steps:
a) culturing the Fc-containing protein in a mammalian cell culture expression system,
b) obtaining the Fc-containing protein composition as a cell culture harvest,
c) subjecting the cell culture harvest comprising a protein mixture to proteolysis to generate fragments of the protein,
d) separating the fragments using liquid chromatography followed by ionization and detection of the fragments in a mass spectrometer,
e) providing unlabeled synthetic peptides homologous to the peptide based impurity and to the native peptide, respectively,
f) confirming the identity of the peptide based impurity using unlabeled synthetic peptide by comparing the retention time and spectral distribution of the peptide mass of the synthetic peptides with the peptide in the protein composition,
g) preparing different dilutions of known concentrations of the unlabeled synthetic peptide and subjecting the said dilutions to liquid chromatography coupled to a mass spectrometer,
h) plotting an area versus concentration graph for the response obtained for known dilutions of the unlabeled synthetic peptides,
i) deducing the absolute amount of native peptide and peptide based impurity using the graph plotted in step (h).

2. A method for identification and absolute quantification of a signal peptide remnant in an Fc-containing protein composition using mass spectrometry, the method comprising the following steps:
a. culturing the Fc-containing protein in a mammalian cell culture expression system,
b. obtaining the Fc-containing protein composition as a cell culture harvest,
c. subjecting the cell culture harvest comprising a protein mixture to proteolysis to generate fragments of the protein,
d. separating the fragments using liquid chromatography followed by ionization and detection of the fragments in a mass spectrometer,
e. providing unlabeled synthetic peptides homologous to the signal peptide remnant and to the native peptide, respectively,
f. confirming the identity of the signal peptide remnant using unlabeled synthetic peptide by comparing the retention time and spectral distribution of the peptide mass of the synthetic peptides with the peptides in the protein composition,
g. preparing different dilutions of known concentrations of the unlabeled synthetic peptide and subjecting the said dilutions to liquid chromatography coupled with a mass spectrometer,
h. plotting an area versus concentration graph for the response obtained for known dilutions of the unlabeled synthetic peptides,
i. deducing the absolute amount of native peptide and signal peptide remnant using the graph plotted in step (h).

3. A method for identification and absolute quantification of a signal peptide remnant in an unpurified or partially purified sample of an Fc-containing protein composition using mass spectrometry, the method comprising the following steps:
a. culturing the Fc-containing protein in a mammalian cell culture expression system,
b. obtaining the Fc-containing protein composition as a cell culture harvest,
c. subjecting the cell culture harvest comprising a protein mixture to proteolysis to generate fragments of the protein,
d. separating the fragments using liquid chromatography followed by ionization and detection of the fragments in a mass spectrometer,
e. providing unlabeled synthetic peptides homologous to the signal peptide remnant and to the native peptide, respectively,
f. confirming the identity of the signal peptide remnant using unlabeled synthetic peptide by comparing the retention time and spectral distribution of the peptide mass of the synthetic peptides with the peptides in the protein composition,
g. preparing different dilutions of known concentrations of the unlabeled synthetic peptide and subjecting the said dilutions to liquid chromatography coupled with a mass spectrometer,
h. plotting an area versus concentration graph for the response obtained for known dilutions of the unlabeled synthetic peptides,
i. deducing the absolute amount of native peptide and signal peptide remnant using the graph plotted in step (h).

4. A method for the identification and absolute quantification of signal peptide remnants in a heterogeneous sample of an Fc-containing protein, using mass spectrometry wherein the method comprises steps of:
a. obtaining a fluid comprising the Fc-containing protein from a mammalian cell culture,
b. filtering the fluid obtained in step (a),
c. obtaining the filtrate of fluid in step (b) comprising a complex mixture of proteins including the Fc-containing protein, host cell proteins, sequence variants, N-terminal signal peptide remnants and subjecting the said filtrate to proteolysis, generating peptide fragments of the proteins in the said filtrate,
d. separating the peptide fragments generated in step (c) using liquid chromatography,
e. providing unlabeled synthetic peptides homologous to the signal peptide remnant and to the native peptide,
f. confirming the identity of the signal peptide remnant using unlabeled synthetic peptide by comparing the retention time and spectral distribution of the peptide mass of the synthetic peptides with the peptide fragments in the Fc-containing protein composition,
g. preparing different dilutions of known concentrations of the unlabeled synthetic peptide and subjecting the said dilutions to liquid chromatography coupled with a mass spectrometer,
h. plotting an area versus concentration graph for the response obtained for known dilutions of the unlabeled synthetic peptides,
i. deducing the absolute amount of native peptide and signal peptide remnant using the graph plotted in step (h).

5. The method as claimed in claim 1, wherein the Fc-containing protein is an Fc-fusion protein.

6. The method as claimed in claim 4, wherein the Fc-fusion protein is selected from the group consisting of etanercept, abatacept, belatacept, alefacept, and aflibercept.

7. The method as claimed in claim 1, wherein the Fc-containing protein is an antibody.

8. The method as claimed in claim 6, wherein the antibody is a therapeutic antibody and is selected from the group consisting of anti-TNF-α antibody, anti-CTLA4 antibody, anti-PD1 antibody, anti-PDL1 antibody, anti-Her2 antibody, anti-IL6R antibody, anti-VEGFR antibody, anti-IL17A antibody, anti-α4β7 antibody, and anti-IgE antibody.

9. The method as claimed in claim 1, wherein the Fc-containing protein is denatured using urea or guanidium hydrochloride.

10. The method as claimed in claim 1, wherein the Fc-containing protein is reduced using dithriothreitol.

11. The method as claimed in claim 1, wherein the reduced Fc-containing protein is alkylated using iodoacetamide.

12. The method as claimed in claim 1, wherein proteolytic digestion of the Fc-region containing protein is performed using trypsin, Lys-C or Glu-c.

13. The method as claimed in claim 1, wherein the digestion solution used for reconstituting the protease comprises 1 M urea, 1 mM EDTA, 20 mM hydroxyl ammonium chloride and 0.1 M Tris and pH of the said solution is about 7.5.

14. The method as claimed in claim 1, wherein the liquid chromatography used to separate the protein fragments is reversed-phase chromatography.

15. The method as claimed in claim 1, wherein the method is capable of detecting signal peptide remnants up to less than 1 ng/μL of the sample.

16. The method as claimed in claim 1, wherein the method is capable of detecting signal peptide remnants up to 0.08 ng/μL of the sample.

17. The method as claimed in claim 1, wherein the method is employed in the early stages of product development for monitoring the level of impurities in-process samples.

18. The method as claimed in claim 2, wherein the method is used to quantify trace-levels of signal peptide remnants in a heterogeneous protein sample comprising a complex mixture of peptides.

19. The method as claimed in claim 2 wherein the method is employed in the early stages of product development for monitoring the level of impurities in-process samples.

20. The method as claimed in claim 3 wherein the method is employed in the early stages of product development for monitoring the level of impurities in-process samples.

21. The method as claimed in claim 3, wherein the method is used to quantify trace-levels of signal peptide remnants in a heterogeneous protein sample comprising a complex mixture of peptides.
